(19) United States
(12) Patent Application Publication
(10) Pub. No.: US 2018/0228596 A1
(43) Pub. Date: Aug. 16, 2018

(54) Device, Kit and Surgical Method Including Sleeve

(71) Applicant: Douglas J. Wyland, Simpsonville, SC (US)

(72) Inventor: Douglas J. Wyland, Simpsonville, SC (US)

(21) Appl. No.: 15/752,657

(22) PCT Filed: Aug. 12, 2016
(86) PCT No.: PCT/US2016/046689

§ 371 Date: Feb. 14, 2018

Related U.S. Application Data
(60) Provisional application No. 62/205,128, filed on Aug. 14, 2015.

(51) Int. Cl. A61F 2/08; A61B 17/04

(52) U.S. Cl. CPC A61F 2/0811; A61B 17/0401; A61F 2/0805; A61B 2017/0404; A61F 2002/0852; A61F 2002/0858; A61F 2002/0864; A61F 2002/0888

(57) ABSTRACT

A device, kit and surgical method are disclosed in which fibrous connective tissues to be attached to bone are protected by a sleeve. Applications exist for ligament, tendon and tenodesis surgeries. The sleeve may be woven, may be tightenable around a distal end of a fibrous connective tissue, and may be formed of non-absorbable materials, absorbable materials combinations thereof. The sleeve may include a biologic material. The sleeve may protect bone cortex regions. A tool may be provided to assist with attachment of the sleeve to the distal end portion. The fibrous connective tissues may be attached to the bone with a screw or a button.

### CROSS-REFERENCE TO RELATED APPLICATIONS

This application is a national stage of International Application No. PCT/US2016/046689, filed Aug. 12, 2016 and claims benefit to U.S. Provisional Patent Application No. 62/205,128, filed Aug. 14, 2015, all of which are incorporated herein by reference herein.

### TECHNICAL FIELD

The present disclosure relates generally to a device, kit and surgical method in which fibrous connective tissue to be attached to bone is protected by a sleeve.

### BACKGROUND

Fibrous connective tissue grafts are used in numerous types of surgical procedures. Anterior cruciate ligament (ACL) and posterior cruciate ligament (PCL) replacement surgeries are but two examples. In such surgeries, patient or donor harvested grafts, or artificial tissue grafts, are attached between the tibia and femur. The grafts may be attached to passages such as sockets or tunnels formed in the tibia and femur. Securing members such as screws or button connectors may be employed to hold the graft in place. U.S. Pat. Nos. 5,139,520 and 8,663,324, for example, describe numerous ACL replacement concepts. The Bio-Tenodesis Screw System, available from Arthrex, Inc., includes a screw and a specialized driver tool for securing grafts in bone sockets. Other soft tissue surgeries are performed on muscles, tendons and ligaments such as the rotator cuff, Achilles tendon, patellar tendon, hips, elbows, shoulders, hands, etc., as noted for example in U.S. Pat. No. 8,834,521.

In surgeries in which graft material is connected to bone, a graft-bone interface area is formed. Depending on the particular surgery, the graft may be held in a passage by a securement device such as a screw or button, and may extend roughly normal to, perpendicular to, or at an intermediate angle to the bone surface. The graft may experience higher forces particularly adjacent the outer edge of the passage (e.g., along the bone cortex later), but also along the entire bone, securement device, and graft interface area.

Certain types of reinforcements have been used in the interface area, both to assist in the secure seating of the screw/button and graft in the passage and to strengthen the graft in this area. For example, a surgeon preparing a graft may add whipstitching along the distal end of the graft for such purposes. The whipstitching may also be employed to add one or more extending sutures to the graft so that the graft can be pulled into and through passage in the bone to properly locate the graft. Adding whipstitching, while conventional and effective, is an extra step during surgery that requires due care and an amount of extra time, tools and material.

Woven elements, such as are shown for example in U.S. Pat. Nos. 8,956,394, 7,729,008, and 6,203,572, have been disclosed for use in surgeries. As noted in these patents, the elements may be used with a screw or graft during the surgery to assist with the location, manipulation or securement of the various elements. However, a need still exists for an improved protective device for a distal end of a fibrous connective tissue that efficiently and effectively avoids certain drawbacks of existing devices and their related surgical methods, and/or provides certain other benefits to the surgeon and patient, such as protection of the distal end and fostering tissue growth after surgery.

### SUMMARY

According to certain aspects of the disclosure, a protective device is disclosed for a fibrous connective tissue having a distal end portion to be secured within a passage in a bone by a securing device. The protective device includes, for example, a sleeve having a first end, a second end, and a tubular portion of a given length between the first and second ends. The first end defines an opening sized for receiving the distal end portion of the fibrous connective tissue into the sleeve. The second end defines an opening sized for receiving a tool for holding the distal end portion of the fibrous connective tissue when the fibrous connective tissue is placed within the sleeve. The length of the sleeve being such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. Various options and modifications are possible.

According to certain other aspects of the disclosure, a protective device is disclosed for a fibrous connective tissue having a distal end portion to be secured within a passage in a bone by a securing device. The protective device includes, for example, a sleeve having a first end, a second end, and a tubular portion of a given length between the first and second ends. The sleeve defines an opening sized for receiving the distal end portion of the fibrous connective tissue into the sleeve. The length of the sleeve is such that when the fibrous connective tissue and sleeve are secured in the passage by the securing device so that the second end of the sleeve extends past an outer opening of the passage just sufficiently so that the sleeve is located between cortex material of the bone and the fibrous connective tissue to thereby protect an interface between the cortex material and the fibrous connective tissue. Various options and modifications are possible.

According to certain other aspects of the disclosure, a protective device is disclosed for a fibrous connective tissue having a distal end portion to be secured within a passage in a bone by a securing device. The protective device includes, for example, a sleeve having a first end, a second end, and a tubular portion of a given length between the first and second ends. The first end defines an opening sized for receiving the distal end portion of the fibrous connective tissue into the sleeve. An eyelet is located at the second end. The length of the sleeve is such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. Various options and modifications are possible.

According to certain other aspects of the disclosure, a protective device is disclosed for a fibrous connective tissue having a distal end portion to be secured within a passage in a bone by a securing device. The protective device includes, for example, a sleeve having a first end, a second end, and a tubular portion of a given length between the first and second ends. The first end defines an opening sized for receiving the distal end portion of the fibrous connective tissue into the sleeve. The length of the sleeve is such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. The sleeve includes a biologic agent. Various options and modifications are possible.

For example, for the protective devices above, the sleeve may be formed of a woven material, and may have a strand weave density sufficient to allow tissue growth between strands after securement in the bone.

The sleeve may have a diameter that decreases in the direction of the second end. The eyelet may include threads that are woven into the sleeve and extend from the first end of the sleeve, and at least one thread may extend from the second end of the sleeve. The sleeve may be configured to tighten around the fibrous connective tissue when the sleeve is extended in a direction along the fibrous connective tissue.

The sleeve may include a non-bioabsorbable material, a bioabsorbable material, or combination of both. The sleeve may include a biologic material, which may be one or more of a collagen matrix, a stem cell and a platelet rich plasma. The sleeve may be sized for use in a tendon surgery and the fibrous connective tissue may be a tendon, or it may be sized for use in a ligament surgery and the fibrous connective tissue may be a ligament.

According to certain other aspects of the disclosure, a surgical kit is disclosed for securing a fibrous connective tissue having a distal end portion within a passage in a bone. The surgical kit may include, for example, a tool for holding the distal end portion of the fibrous connective tissue and a sleeve having a first end, a second end, and a tubular portion of a given length between the first and second ends. The first end defines an opening sized for receiving the distal end portion of the fibrous connective tissue into the sleeve. The second end defines an opening sized for receiving the tool when the fibrous connective tissue is placed within the sleeve. The length of the sleeve being such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. A securing device is provided for securing the distal end portion and the sleeve to the passage. Various options and modifications are possible.

According certain other aspects of the disclosure, a surgical kit is disclosed for securing a fibrous connective tissue having a distal end portion within a passage in a bone. The surgical kit may include, for example, a tool for holding the distal end portion of the fibrous connective tissue and a sleeve having a first end, a second end, and a tubular portion of a given length between the first and second ends. The first end defines an opening sized for receiving the distal end portion of the fibrous connective tissue into the sleeve. The length of the sleeve is such that when the fibrous connective tissue and sleeve are secured in the passage by the securing device the second end of the sleeve extends past an outer opening of the passage just sufficiently so that the sleeve is located between cortex material of the bone and the fibrous connective tissue to thereby protect an interface between the cortex material and the fibrous connective tissue. A securing device is provided for securing the distal end portion and the sleeve to the passage. Various options and modifications are possible.

According to certain other aspects of the disclosure, a surgical kit is disclosed for securing a fibrous connective tissue having a distal end portion within a passage in a bone. The surgical kit may include, for example, a tool for holding the distal end portion of the fibrous connective tissue and a sleeve having a first end, a second end, and a tubular portion of a given length between the first and second ends. The first end defines an opening sized for receiving the distal end portion of the fibrous connective tissue into the sleeve. An eyelet is located at the second end. The length of the sleeve is such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. A securing device is provided for securing the distal end portion and the sleeve to the passage. Various options and modifications are possible.

According to certain other aspects of the disclosure, a surgical kit is disclosed for securing a fibrous connective tissue having a distal end portion within a passage in a bone. The surgical kit may include, for example, a tool for holding the distal end portion of the fibrous connective tissue and a sleeve having a first end, a second end, and a tubular portion of a given length between the first and second ends. The first end defines an opening sized for receiving the distal end portion of the fibrous connective tissue into the sleeve. The length of the sleeve is such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue. The sleeve includes a biologic agent. A securing device is provided for securing the distal end portion and the sleeve to the passage. Various options and modifications are possible.

For example, with the kits above, the sleeve may be formed of a woven material, which may have a strand weave density sufficient to allow tissue growth between strands after securement in the bone.

The sleeve may have a diameter that decreases in the direction of the second end. The eyelet may include threads that are woven into the sleeve and extend from the first end of the sleeve, and at least one thread may extend from the second end of the sleeve. The sleeve may be configured to tighten around the fibrous connective tissue when the sleeve is extended in a direction along the fibrous connective tissue.

The sleeve may include a non-bioabsorbable material, a bioabsorbable material, or combinations of both. The sleeve may include a biologic material, which may be one or more of a collagen matrix, a stem cell and a platelet rich plasma.

The sleeve may be sized for use in a tendon surgery and the fibrous connective tissue may be a tendon, or it may be sized for use in a ligament surgery and the fibrous connective tissue may be a ligament.

The securing device may be a screw and/or a button. The kit may include two of the sleeves and two of the securing devices, and may include a movable gripper for gripping the distal end portion. The tool may also include a fixed extension for holding the distal end portion.

According to certain other aspects of the disclosure, a surgical method is disclosed for securing a fibrous connective tissue having a distal end portion within a passage in a bone. The surgical method may include, for example, holding the distal end portion of the fibrous connective tissue with a tool extending through a sleeve, the sleeve having a first end, a second end, and a tubular portion between the first and second ends, the sleeve defining a length between the first and second ends; placing the sleeve over the distal end portion; removing the tool from the sleeve; inserting the fibrous connective tissue and sleeve into the passage so that the sleeve is located between cortex material of the bone and the fibrous connective tissue to thereby protect an interface between the cortex material and the fibrous connective tissue; and securing the fibrous connective tissue and sleeve to the passage. Various options and modifications are possible.

According to certain other aspects of the disclosure, a surgical method is disclosed for securing a fibrous connective tissue having a distal end portion within a passage in a bone. The surgical method may include, for example, holding the distal end portion of the fibrous connective tissue with a tool; placing a sleeve over the distal end portion, the sleeve having a first end, a second end, and a tubular portion between the first and second ends, the sleeve defining a length between the first and second ends; inserting the fibrous connective tissue and sleeve into the passage just sufficiently so that the sleeve is located between cortex material of the bone and the fibrous connective tissue to thereby protect an interface between the cortex material and the fibrous connective tissue; and securing the fibrous connective tissue and sleeve to the passage. Various options and modifications are possible.

According to certain other aspects of the disclosure, a surgical method is disclosed for securing a fibrous connective tissue having a distal end portion within a passage in a bone. The surgical method may include, for example, holding the distal end portion of the fibrous connective tissue with a tool; placing a sleeve over the distal end portion, the sleeve having a first end, a second end, and a tubular portion between the first and second ends, the sleeve defining a length between the first and second ends, an eyelet being located at the second end; inserting the fibrous connective tissue and sleeve into the passage at least partially by pulling the eyelet so that the sleeve is located between cortex material of the bone and the fibrous connective tissue to thereby protect an interface between the cortex material and the fibrous connective tissue; and securing the fibrous connective tissue and sleeve to the passage. Various options and modifications are possible.

According to certain other aspects of the disclosure, a surgical method is disclosed for securing a fibrous connective tissue having a distal end portion within a passage in a bone. The surgical method may include, for example, holding the distal end portion of the fibrous connective tissue with a tool; placing a sleeve over the distal end portion, the sleeve having a first end, a second end, and a tubular portion between the first and second ends, the sleeve defining a length between the first and second ends, threads woven into the sleeve extending from the first end; tightening the sleeve around the distal end portion by pulling the threads; inserting the fibrous connective tissue and sleeve into the passage so that the sleeve is located between cortex material of the bone and the fibrous connective tissue to thereby protect an interface between the cortex material and the fibrous connective tissue; and securing the fibrous connective tissue and sleeve to the passage. Various options and modifications are possible.

For example, the method above may further include the step of attaching the sleeve to the fibrous connective tissue before the inserting step. The attaching step may include pulling threads on the sleeve to tighten the sleeve around the fibrous connective tissue.

The method may further include stitching the sleeve to the fibrous connective tissue. The securing step may include placing a screw into the passage and/or attaching a button fastener through the passage.

The method may further include repeating the holding, placing, inserting and securing steps with a second sleeve, a second distal end portion of the fibrous connective tissue, and a second bone. The first bone may be one of a femur or tibia and the second bone may be the other of a femur or tibia. The passage may be a socket or a tunnel.

The fibrous connective tissue may be a tendon or a ligament. The fibrous connective tissue may be an autograft, an allograft, a heterograft or an artificial tissue.

The method may include intra-articular surgery, an extra-articular surgery or tenodesis surgery.

The sleeve may be formed of a woven material, and it may have a strand weave density sufficient to allow tissue growth between strands after securement in the bone. The sleeve may include a non-bioabsorbable material, a bioabsorbable material, or combinations of both. The sleeve may include a biologic material, which may be one or more of a collagen matrix, a stem cell and a platelet rich plasma.

The tool may include a movable gripper for gripping the distal end portion, and may include a fixed extension for holding the distal end portion.

With all of the above sleeves, the sleeves may be self-locking so that once tightened on tissue, the sleeves substantially maintain their grip on the tissue.

Also, the sleeves may have a varying weave density along their lengths. If so, the weave density may be lower along the portion inserted into the socket or tunnel of bone and may be higher along the portion along the bone surface (outer cortex).

### BRIEF DESCRIPTION OF THE DRAWINGS

More details of the present disclosure are set forth in the drawings.

FIG. 1 is a perspective view of one example of a protective device according to certain aspects of the disclosure.

FIG. 2 is a perspective view of the protective device of FIG. 1 as secured to the distal end of a fibrous connective tissue.

FIG. 3 is a diagrammatic sectional view of the protective device and fibrous connective tissue as in FIG. 2, as secured in a passage of a bone via a screw.

FIG. 4 is a diagrammatic view of the protective device and fibrous connective tissue as in FIG. 2, as secured in a passage of a bone via a button.

FIG. 5 is a diagrammatic sectional view of the protective device and fibrous connective tissue as in FIG. 2, as secured in a passage of a bone via a screw at an angle different than in FIG. 2.

FIG. 6 is a diagrammatic view of two of the protective devices and a fibrous connective tissue as in FIG. 2, as secured in a knee joint via two screws.

FIGS. 7-12 are perspective diagrammatic views showing one set of steps of a surgical method using the protective device and fibrous connective tissue as in FIG. 2.

FIGS. 13-18 are perspective views showing alternate configurations for protective devices.

### DETAILED DESCRIPTION

Detailed reference will now be made to the drawings in which examples embodying the present disclosure are shown. The detailed description uses numeral and letter designations to refer to features in the drawings. Like or similar designations in the drawings and description have been used to refer to like or similar parts of the disclosure.

The drawings and detailed description provide a full and enabling description of the disclosure and the manner and process of making and using it. Each embodiment is provided by way of explanation of the subject matter not limitation thereof. In fact, it will be apparent to those skilled in the art that various modifications and variations may be made to the disclosed subject matter without departing from the scope or spirit of the disclosure. For instance, features illustrated or described as part of one embodiment may be used with another embodiment to yield a still further embodiment.

Generally speaking, FIGS. 1-18 show examples of different constructs, kits and surgical steps for a protective device that can be used for securing fibrous connective tissue to a passageway in a bone. It should be understood that the fibrous connective tissues may be any such tissue suitable for surgical repair now or in the future, such as ligaments, tendons, etc. The fibrous connective tissues may be made of any sort suitable substance, such as an autograft, allograft, heterograft, or artificial tissue, etc. The fibrous connective tissues may be formed in many ways, including single strand, multiple strands, folded strands, linear or branched constructs, stitched, unstitched, etc.

Also, the bone may be any bone suitable for surgical connective fibrous tissue repair, such as a tibia, femur, or humerus, but also any other bone within the body. The surgical procedure may for example be intra-articular, extra-articular or tenodesis procedures. The passages formed in the bone may be in the form of a conventional socket, tunnel, or the like. The fibrous connective tissues may be attached to the passages in the bones with securement devices such as conventional screws, screws with swivel lock anchors, button devices, etc. The surgical procedures and locations are not limited to the human body and may include use with animal species.

Thus, it should be understood that the protective devices, kits, and surgical procedures disclosed herein have applicability beyond the examples disclosed below.

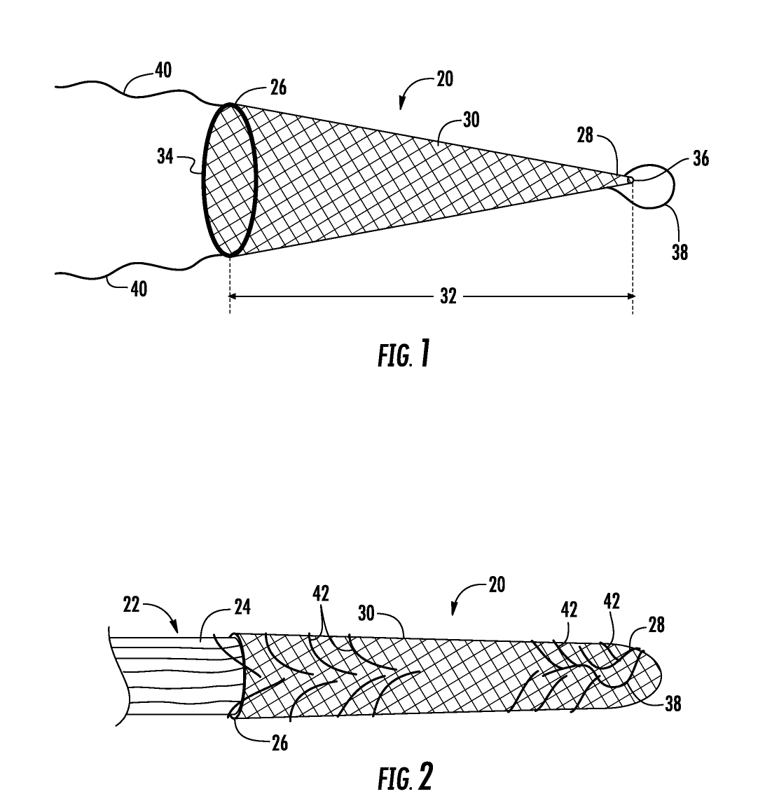

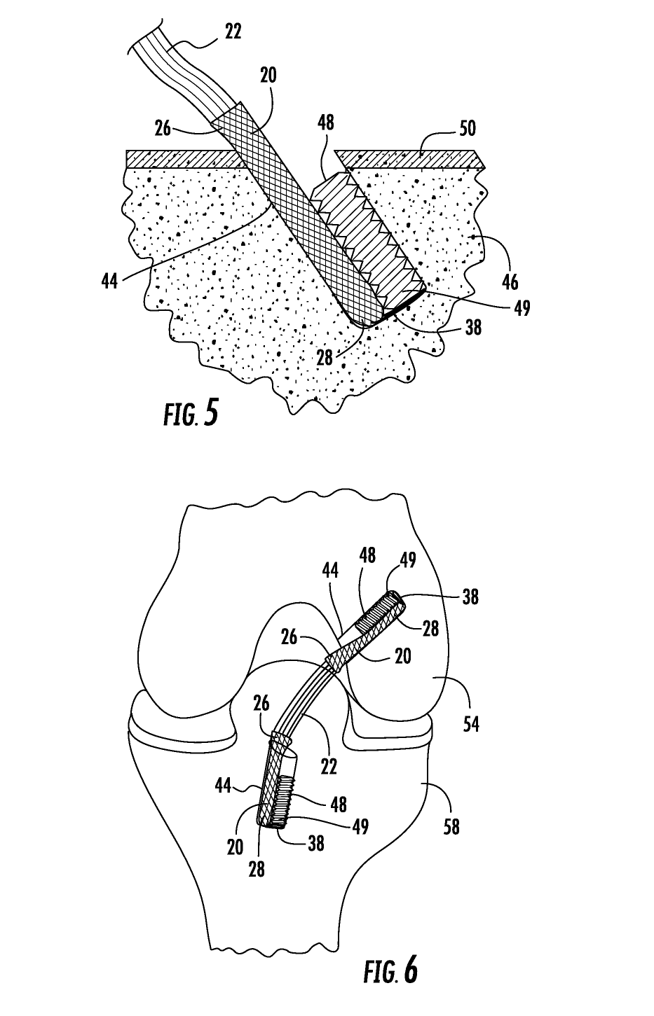

More particularly, FIGS. 1-2 show an example of a protective device in the form of a sleeve 20 for use with a fibrous connective tissue 22 having a distal end portion 24. Sleeve 20 has a first end 26, a second end 28, and a tubular portion 30 of length 32 between the first and second ends. First end 26 defines an opening 34 sized for receiving distal end portion 24 of fibrous connective tissue 22 into the sleeve. Second end 28 defines an opening 36 sized for receiving a tool (see FIG. 7) for holding distal end portion 24 when fibrous connective tissue 22 is placed within the sleeve 20. It should be understood, however, that opening 36 is not required in all facets of the disclosure. Sleeve 20 is preferably configured so that length 32 of the sleeve 20 will cover and therefore protect the portion of fibrous connective tissue 22 within the sleeve while and after the tissue is secured in a bone passage, such as a socket or tunnel, as described below.

Sleeve 20 is formed of a woven material. Sleeve 20 is flexible and pliable so that it can be manipulated, stretched, tightened, etc., as needed during a procedure. The woven material may have a thread weave density sufficient to allow tissue growth between strands after securement in the bone. Sleeve 20 can be formed of various materials, either non-bioabsorbable and bioabsorbable, or combinations of both. Thus, different types and sizes of strands can be woven into sleeve 20 to provide different properties. The woven material can also be coated by or infused with a biologic material suitable for various purposes, such as one or more of a collagen matrix, a stem cell and a platelet rich plasma.

As shown in FIG. 1, sleeve 20 has a diameter that decreases in the direction of second end 28. FIG. 1 shows the decreasing diameter as a somewhat constant taper. However, other shapes are possible, as mentioned below.

An eyelet 38 may be included at second end 28. Eyelet 38 may be formed of threads that are woven into sleeve 20. Other threads 40 woven into sleeve 20 may extend from first end 26. Alternately, threads 40 and eyelet 38 may be formed of the same threads.

Sleeve 20 may be configured to tighten around fibrous connective tissue 22 when the sleeve is extended in a direction along the fibrous connective tissue. Thus, pulling longitudinally on sleeve 20 tightens and thins its weave, in some ways like a finger trap. In particular, pulling on threads 40 and/or eyelet 38 while holding sleeve 20 on fibrous connective tissue distal end portion 24 provides a locking attachment of the sleeve to the tissue. Sleeve 20 can be described as “self-locking” if after pulling on one or both of the threads 40 the sleeve 20 substantially retains its shape and grip on tissue 22. Depending on the surgeon's preference, such locking attachment may be used to hold sleeve 20 on tissue 22 until a screw is used to attach the sleeve and tissue to the bone. If desired, after threads 40 are pulled, the threads can be threaded back into the mesh of sleeve 20, tied as a secondary lock, or used as sutures (e.g., as a whipstitch) to further secure the sleeve to tissue 22.

Additional sutures 42, such as one or more stitches, whipstitching or other patterns, may also be used if it's the surgeon's preference to further secure sleeve 20 to the fibrous connective tissue once the sleeve is secured by the initial tightening (see FIG. 2). Sutures 42 may be formed from threads 40, or may be in addition to threads 40, and may be used to secure both ends 26, 28 of sleeve 20 to tissue 22. Such sutures 42 may also secure eyelet 38 to sleeve 20 after placement of sleeve on tissue 22 (as shown in FIG. 2), or eyelet 38 may be left to extend from sleeve (as shown in FIG. 1).

FIGS. 3-6 generally show examples of securement of a fibrous connective tissue graft to a passage in a bone using a securing device and a sleeve. FIG. 3 shows such an arrangement of distal end portion 24 of tissue 22, within sleeve 20, secured in a passage 44 in a bone 46 via a screw 48. As shown, passage 44 is a socket formed normally to the bone surface (outer cortex portion 50). Sleeve 20 extends out of passage just past outer cortex portion 50. Sleeve 20 therefore protects the graft during insertion of screw 48, during any surgical repositioning, and afterwards. In particular, sleeve 20 protects the interface between the edge of passage 44 along the cortex portion 50. As ligaments and tendons naturally exist to allow relative movement, they are subjected to various forces as a patient moves. Protection of the interface may be important, because a somewhat sharp edge may exist, and because forces may be transmitted at a sharp angle (see dotted line orientation in FIG. 3).

However, in the examples herein, the entire fibrous connective tissue graft 22 is not covered by sleeve 20. Central portions of the graft (between bone securement sites) would not necessarily need protection and reinforcement from bone and bone edge contact. Also, by only covering ends of the graft with sleeves, less material is used leading to a less bulky construct, which may be more convenient for handling during surgery, in particular arthroscopic surgery. Also, more options are available as to sizing by only covering ends; sleeves of different sizes may be used on different ends, and a family of differently sized sleeves may be provided for selection by the surgeon as needed.

By way of example, if passage 44 were a socket with a depth of 25 mm, sleeve 20 could have a longer length, e.g., 30 or 35 mm, so as to extend past cortex portion 50 along the outer surface of bone 46. Other dimensions and rations of such dimensions are possible, depending on the surgical site, surgeon preference, etc.

Also, the length and during- and post-surgical orientation of tissue 22, sleeve 20, and screw 48 may vary, which may also have an effect on the length of sleeve vs. depth of passage ratio. Thus, as shown in FIG. 3, no portion of tissue 22 or sleeve is located along a bottom end 49 of screw 48. If end 28 of sleeve 20 on tissue 22 were to be held beneath bottom end 49 of screw 48 (rather than being located adjacent bottom end 49 of screw 48), then the sleeve used could be longer (relative to the depth of passage 44) so that end 26 of sleeve 20 still extends past cortex portion 50. Thus, and extra 5 or 10 mm of length could be added to sleeve 20 if it is to be inserted in such fashion, as compared to the dimensions set forth above.

If a tool with a gripping end portion, hook, loop, etc., is employed to insert screw 48, then such gripping element can engage eyelet 38. By doing so, the tissue 22 and sleeve 20 can be pulled into passage 44 in a desired orientation (either linearly as shown in Fib. 3 or with end 28 of sleeve 20 and tissue 22 beneath end 49 of screw 48, as described above). Such engagement of eyelet 28 can occur whether or not eyelet 38 has been stitched to sleeve/tissue as shown in FIG. 2. If a tool such as The Bio-Tenodesis System is employed, the distal end of the tool can contact and pull eyelet 38 and/or stitching 42 to place tissue 22 and sleeve 20 in passageway 44 before screw 48 is driven into place.

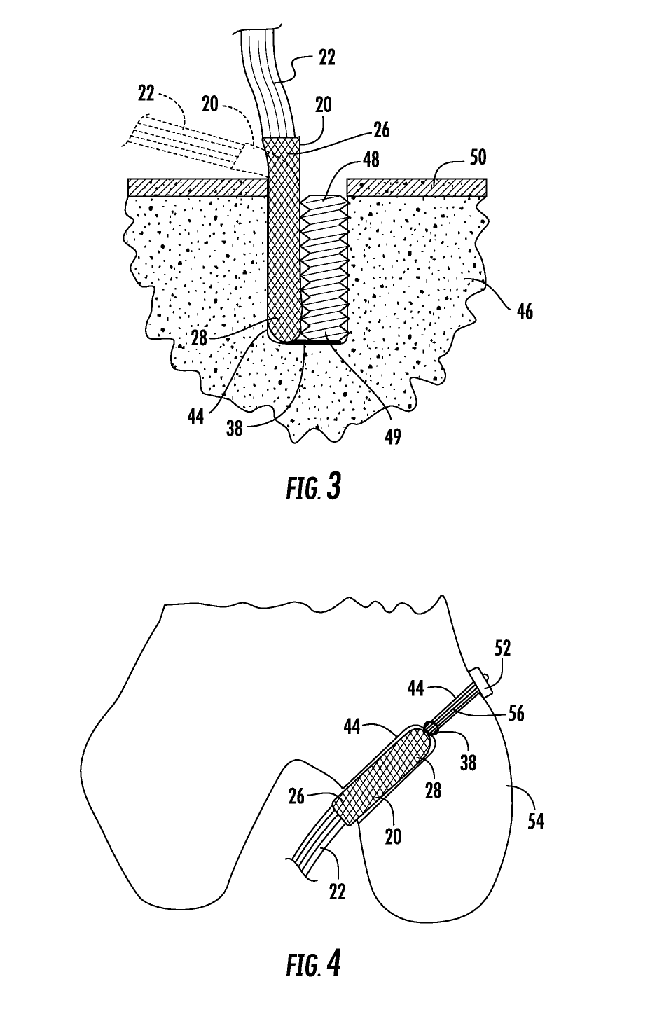

FIG. 4 shows sleeve 20 and fibrous connective tissue 22 connected to a tunnel-type passage extending through a bone such as a femur 54 by a conventional button fastener 52 and strands 56 attached to the sleeve and tissue construct. Sleeve 20 convers distal end portion 24 and extends just past the surface of bone 52, as above. Distal end portion 24 and sleeve 20 are connected to strands 56 by conventional stitching (not shown for clarity). A screw (not shown) can be used to further secure the sleeve and tissue in the tunnel.

FIG. 5 shows a screw-secured arrangement as in FIG. 3, but where passage 44 is a socket drilled in bone 46 at an angle to cortex portion 50.

FIG. 6 shows a knee joint including a femur 54 and tibia 58, in which a fibrous connective tissue 22 has been secured in two sockets 44 by screws 48. Sleeves 20 are located at each end of tissue 22, and extend just past the respective edges of the sockets 44, as above.

It should be understood that the above examples of tissue and bone are exemplary and non-limiting.

FIGS. 7-12 show one method of placement of a sleeve on a tissue, using a tool. As illustrated, tool 60 includes two gripping arms 62 attached to a central portion 64, one or both of which may be relatively movable toward and away from the other, generally in the form of a forceps, clamp, etc. The handle and operating mechanism of tool 60 need not be shown for purposes of this disclosure and could have many known forms.

In FIG. 7, tool 60 is supplied with sleeve 20 placed over tool 60 in a rolled configuration so that central portion 64 extends through opening 36. Alternately, sleeve 20 can be slid onto tool 60 without rolling. Sleeve 20 is rolled into a small configuration and exposes gripping members 62 sufficiently that they can grip tissue 22. Gripping members 62 are opened, if necessary, to receive tissue 22 then closed to lightly clamp tissue 22, as shown in FIG. 8.

In FIG. 9, sleeve 20 has been unrolled to cover part of tissue 22 and more of tool 60. Note eyelet 38 and threads 40 (if present) would be exposed after unrolling.

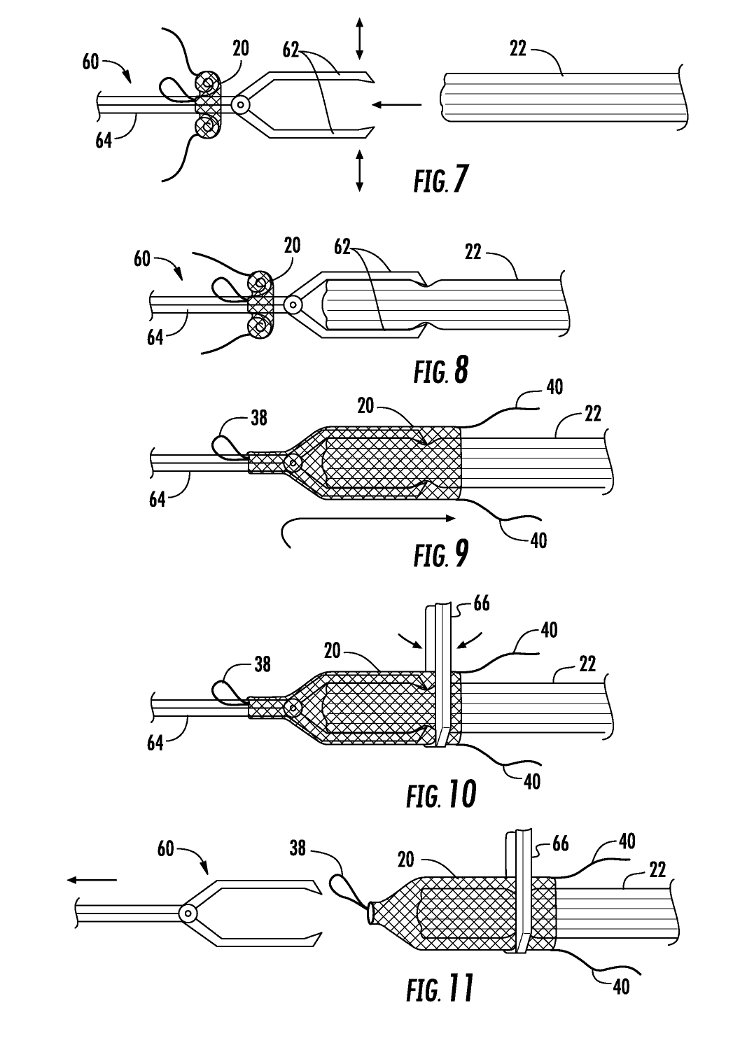

In FIG. 10, optionally a hemostat clamp 66 or the like may be employed to hold sleeve 20 on tissue 22 so that tool 60 can be removed. Accordingly, gripping members 62 release the tissue 22 at this point.

In FIG. 11, tool 60 has been slid out of sleeve 20 via opening 36. Note that the flexibility of the weave allows the tool to move out of sleeve 20 without damaging the sleeve.

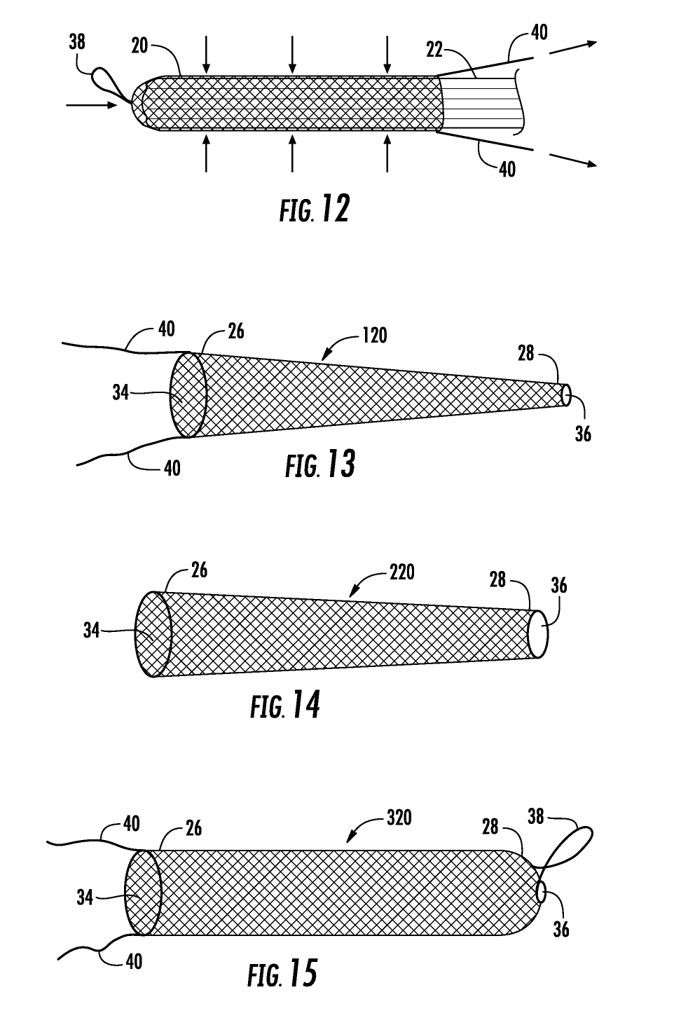

In FIG. 12, clamp 66 has been removed, and threads 40 have been pulled to lengthen sleeve 20 and tighten it around tissue 22. If desired, a clamp could be used in this step adjacent second end 36 while pulling on threads 40. If desired, eyelet 38 can use used to hold the second end, either directly or with a tool, during such pulling. The pulling of the woven sleeve tightens and preliminarily locks (and/or self-locks) it on distal end portion 24 of tissue 22. Suturing can then be added as desired to hold sleeve 20 to tissue 22, for example, to engage with bone screws, to assist in mounting a button securing structure, as shown in FIG. 2. Eyelet 38 can be employed as some or part of the button mounting structure, if desired.

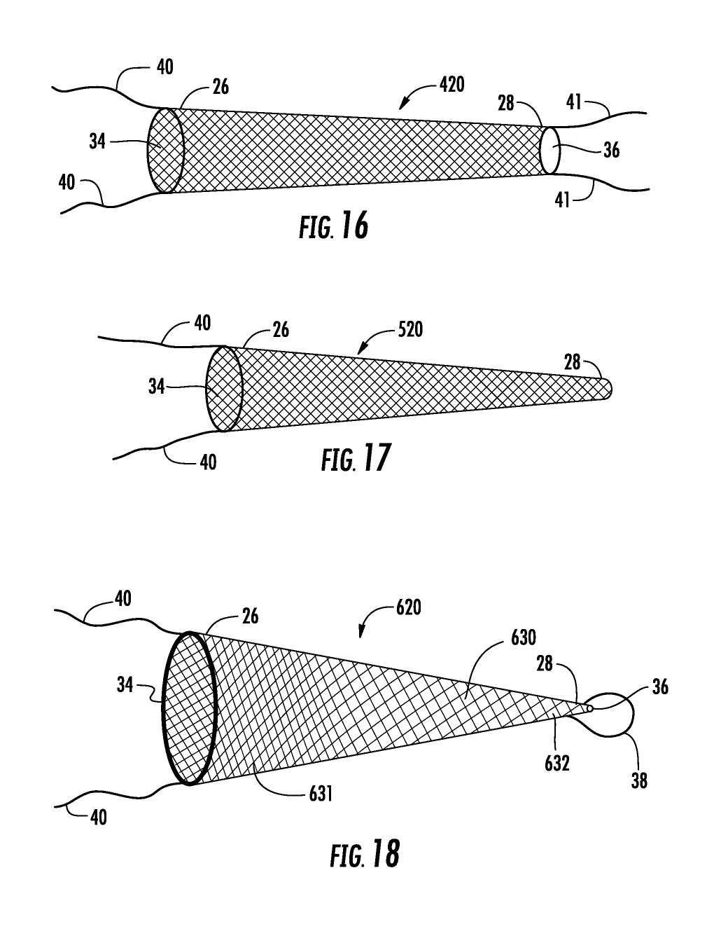

Alternate sleeve designs are shown in FIGS. 13-18. The alternate designs may be similar to sleeve 20, except for the differences noted below. For example, sleeve 120 in FIG. 13 is similar to sleeve 20, but does not include an eyelet. Sleeve 220 in FIG. 14 further excludes the threads 40 of sleeve 20. Sleeve 320 in FIG. 15 is substantially of constant diameter from first end 26 to second end 28 and only has taper near the second end. Sleeve 420 in FIG. 16 replaces eyelet 38 of sleeve 20 with additional threads 41 at second end 28. Sleeve 520 in FIG. 17 does not include the opening 36 found in second end 28 of sleeve 20.

Sleeve 620 in FIG. 18 has a relatively denser mesh at portion 631 extending from first end 26 and a relatively less dense mesh at portion 632 extending from second end 28. The first portion 631 may be of lesser length (between 26 and 28) then the second portion 632. If so, first portion 631 may have a ratio of 1:2, 1:3, 1:4, etc. relative to the second portion. Alternatively, first portion 631 may have a given length (e.g. 5 mm, 10 mm, etc.) and second portion 632 may comprise the rest of sleeve 620. The denser mesh provides more protection in the area around the edge of passage 44 along the cortex portion 50. The less dense mesh provides more area for contract between tissue 22 and the bone to promote healing and attachment.

A kit may be provided including one or more sleeves 20-520 of one or more sizes, one or more tools 60, and one or more securing members such as screws and/or button connectors. Sleeves may be provided in different diameters, for example such as 3, 5, and 7 mm, with different lengths for example such as 10, 15, 20, 25 and 30 mm, to provide a surgeon with options during surgery.

Accordingly, a device, kit and surgical method are disclosed in which a sleeve is used to protect a distal end of a fibrous connective tissue. While preferred embodiments of the invention have been described above, it is to be understood that any and all equivalent realizations of the present invention are included within the scope and spirit thereof. Thus, the embodiments depicted are presented by way of example only and are not intended as limitations upon the present invention. Thus, while particular embodiments of the invention have been described and shown, it will be understood by those of ordinary skill in this art that the present invention is not limited thereto since many modifications can be made. Therefore, it is contemplated that any and all such embodiments are included in the present invention as may fall within the literal or equivalent scope of the appended claims.

1. A protective device for a fibrous connective tissue having a distal end portion to be secured within a passage in a bone by a securing device, the protective device comprising:
a sleeve having a first end, a second end, and a tubular portion between the first and second ends, the sleeve defining a length between the first and second ends, the first end defining an opening sized for receiving the distal end portion of the fibrous connective tissue into the sleeve, the second end defining an opening sized for receiving a tool for holding the distal end portion of the fibrous connective tissue when the fibrous connective tissue is placed within the sleeve, the length of the sleeve being such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue.

2-17. (canceled)

18. A surgical kit for securing a fibrous connective tissue having a distal end portion within a passage in a bone, the surgical kit comprising:
a tool for holding the distal end portion of the fibrous connective tissue;
a sleeve having a first end, a second end, and a tubular portion between the first and second ends, the sleeve defining a length between the first and second ends, the first end defining an opening sized for receiving the distal end portion of the fibrous connective tissue into the sleeve, the second end defining an opening sized for receiving the tool when the fibrous connective tissue is placed within the sleeve, the length of the sleeve being such that when the fibrous connective tissue and sleeve are secured in the passage by the securing device the second end of the sleeve extends past an outer opening of the passage so that the sleeve is located between cortex material of the bone and the fibrous connective tissue to thereby protect an interface between the cortex material and the fibrous connective tissue; and
a securing device for securing the distal end portion and the sleeve to the passage.

19-39. (canceled)

40. A surgical method of securing a fibrous connective tissue having a distal end portion within a passage in a bone, the surgical method comprising:
holding the distal end portion of the fibrous connective tissue with a tool extending through a sleeve, the sleeve having a first end, a second end, and a tubular portion between the first and second ends, the sleeve defining a length between the first and second ends;
placing the sleeve over the distal end portion;
removing the tool from the sleeve;
inserting the fibrous connective tissue and sleeve into the passage so that the sleeve is located between cortex material of the bone and the fibrous connective tissue to thereby protect an interface between the cortex material and the fibrous connective tissue; and
securing the fibrous connective tissue and sleeve to the passage.

41-71. (canceled)
